(19) United States
(12) Patent Application Publication
(10) Pub. No.: US 2024/0058071 A1
(43) Pub. Date: Feb. 22, 2024

(54) LEFT ATRIAL APPENDAGE CLOSURE PRE-PROCEDURE SYSTEM AND METHODS

(71) Applicant: BOSTON SCIENTIFIC SCIMED, INC., Maple Grove, MN (US)

(72) Inventors: Jan-Hung Chen, St. Paul, MN (US); Harishankar Natesan, Minneapolis, MN (US); Joseph Walker, Shoreview, MN (US); Cory Swingen, New Brighton, MN (US)

(73) Assignee: BOSTON SCIENTIFIC SCIMED, INC., Maple Grove, MN (US)

(21) Appl. No.: 18/450,934

(22) Filed: Aug. 16, 2023

Related U.S. Application Data
(60) Provisional application No. 63/398,772, filed on Aug. 17, 2022.

(51) Int. Cl. A61B 34/10; G16H 20/40; G06F 30/20; G06T 17/00; G06T 7/62

(52) U.S. Cl. CPC A61B 34/10; G16H 20/40; G06F 30/20; G06T 17/00; G06T 7/62; G06T 2210/41; G06T 2207/30101; A61B 2034/104; A61B 2034/105

(57) ABSTRACT

This disclosure teaches determining a recommended LAA closure device size based on simulations of deployment of multiple closure devices with the LAA anatomy. Input images of the patient's LAA anatomy are received. Multiple simulations are run providing options of device interaction, closure, and anchoring for multiple anchoring sites and device sizes. Gaps between the simulated deployed devices are analyzed based on leak paths and a recommended closure device size determined based on the simulated gaps.

### CROSS-REFERENCE TO RELATED APPLICATIONS

This application claims the benefit of priority under 35 U.S.C. § 119 of U.S. Provisional Application No. 63/398,772, filed Aug. 17, 2022, the entire disclosure of which is hereby incorporated by reference.

### TECHNICAL FIELD

The present disclosure generally relates to left atrial appendage (LAA) closure procedures. Particularly, but not exclusively, the present disclosure relates to pre-planning LAA closure device size and placement.

### BACKGROUND

Human left atrial appendages (LAAs) are highly variable in size and shape from patient to patient, which makes it challenging for physicians to determine the appropriate LAA closure device size and deployment position. Computed tomography (CT) based pre-planning allows physicians to assess a patient's LAA ahead of a procedure. However, available CT based pre-planning tools provide limited information. Thus, there is a need for more advanced pre-planning tools than conventionally available.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides LAA closure pre-planning methods and systems that address shortcomings in conventional solutions. For example, the pre-planning methods and systems according to the present disclosure can provide critical information to a physician, such as, for example, the interaction between the closure device and the LAA, sealing of the closure device, anchoring of the closure device, etc.

In general, the present disclosure provides to receive as input images of the patient's LAA anatomy (e.g., from CT based pre-procedure images, or the like). Multiple simulations are run providing options of device interaction, closure, and anchoring for multiple anchoring sites and device sizes. With some examples, the simulation can provide a recommendation for an ideal closure device size based on simulated LAA interaction, sealing, and anchoring results.

In some examples, the present disclosure provides a method of pre-planning a left atrial appendage (LAA) closure; the method comprising: receiving images comprising indication of an LAA of a patient; receiving an indication of a landing zone of a closure device; simulating a deployment of the closure device in the LAA of the patient; simulating a sealing of the deployed closure device against the LAA of the patient; simulating an anchoring of the closure device in the LAA of the patient; and generating a graphical representation of the sealing and anchoring of the closure device in the LAA of the patient.

The method may further comprise generating a graphical representation of the deployed closure device in the LAA of the patient. In some implementations, generating a graphical representation of the deployed closure device in the LAA of the patient comprises overlaying the simulated deployed closure device with a graphical representation of the LAA of the patient. The method may further comprise determining a recommended size of the closure device based on the received images. The method may further comprise identifying a diameter of the ostium of the LAA of the patient based on the received images; selecting from a set of closure device sizes a closure device size within a threshold expanded diameter to the diameter of the ostium; and identifying the selected closure device size as the recommended closure device. The method may further comprise simulating the deployment of the closure device in the LAA of the patient for a closure device having the recommended size. The method may further comprise simulating a deployment of the closure device in the LAA of the patient for a closure device having a size different from the recommended size. The method may further comprise generating a graphical representation of the deployed closure device in the LAA of the patient comprising overlaying the simulated deployed closure device having the recommended size with the graphical representation of the LAA of the patient and overlaying the simulated deployed closure device having a different size with the graphical representation of the LAA of the patient. In some implementations, generating the graphical representation of the sealing of the deployed closure device in the LAA of the patient comprises generating a plurality of images comprising an indication of the LAA of the patient and an indication of a contact surface, the contact surface representative of a contact between the deployed closure device and the LAA of the patient. With some implementations, the plurality of images comprises the indication of the LAA of the patient, the indication of the contact surface and an indication of a leak path. The method may further comprise generating a rotatable model from the plurality of images. In some implementations, generating the graphical representation of the anchoring of the closure device in the LAA of the patient comprising generating an image of the simulated deployed closure device comprising indications of anchors of the closure device and a representation of an engagement of the anchors with tissue of the LAA of the patient. With some implementations, the representation of the engagement of the anchors comprises a color map.

In some embodiments, the present disclosure can be implemented as a computer readable storage medium comprising instructions, which when executed by a processor of a computing device cause the processor to implement any of the methods described herein. With some embodiments, the present disclosure can be implemented as a computer comprising a processor; and memory comprising instructions, which when executed by the processor cause the computing system to implement any of the methods described herein.

In some examples, the present disclosure can be implemented as a computing system, comprising: a processor; and memory comprising instructions, which when executed by the processor cause the computing system to: receive images comprising indication of an LAA of a patient; receive an indication of a landing zone of a closure device; simulate a deployment of the closure device in the LAA of the patient; simulate a sealing of the deployed closure device against the LAA of the patient; simulate an anchoring of the closure device in the LAA of the patient; and generate a graphical representation of the sealing and anchoring of the closure device in the LAA of the patient.

In some implementations, the computing system includes the instruction, which when executed by the processor, cause the computing system to generate a graphical representation of the deployed closure device in the LAA of the patient. With some implementations, the computing system includes the instruction, which when executed by the processor, cause the computing system to generate a graphical representation of the deployed closure device in the LAA of the patient comprising overlaying the simulated deployed closure device with a graphical representation of the LAA of the patient. In some implementations, the computing system includes the instruction, which when executed by the processor, cause the computing system to determine a recommended size of the closure device based on the received images.

With some examples, the disclosure includes a computer readable storage medium comprising instructions, which when executed by a processor of a computing device cause the processor to receive images comprising indication of an LAA of a patient; receive an indication of a landing zone of a closure device; identifying a diameter of the ostium of the LAA of the patient based on the received images; selecting from a set of closure device sizes a closure device size within a threshold expanded diameter to the diameter of the ostium; identifying the selected closure device size as the closure device; simulate a deployment of the closure device in the LAA of the patient; simulate a sealing of the deployed closure device against the LAA of the patient; simulate an anchoring of the closure device in the LAA of the patient; and generate a graphical representation of the sealing and anchoring of the closure device in the LAA of the patient.

In some embodiments, the computer readable storage medium includes instruction, which when executed by the processor, cause the computing system to simulate the deployment of the closure device in the LAA of the patient for a closure device having the recommended size. With some embodiments, the computer readable storage medium includes instruction, which when executed by the processor, cause the computing system to simulate a deployment of the closure device in the LAA of the patient for a closure device having a size different from the recommended size.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates LAA closure planning system, in accordance with embodiment(s) of the present disclosure.

FIG. 2 illustrates a logic flow, in accordance with embodiment(s) of the present disclosure.

FIG. 3A illustrates images of an LAA, in accordance with embodiment(s) of the present disclosure.

FIG. 3B illustrates images of an LAA, in accordance with embodiment(s) of the present disclosure.

FIG. 4A and FIG. 4B illustrate a simulated sealing of a closure device, in accordance with embodiment(s) of the present disclosure.

FIG. 5A, FIG. 5B, FIG. 5C, and FIG. 5D illustrate a leak path, in accordance with embodiment(s) of the present disclosure.

FIG. 6A, FIG. 6B, and FIG. 6C illustrate a simulates sealing of a closure device, in accordance with embodiment(s) of the present disclosure.

FIG. 7 illustrates a leak path, in accordance with embodiment(s) of the present disclosure.

FIG. 8 a graph of the leak path, in accordance with embodiment(s) of the present disclosure.

FIG. 9A and FIG. 9B illustrate a simulated anchor engagement, in accordance with embodiment(s) of the present disclosure.

FIG. 10 illustrates an example computer readable storage medium, in accordance with embodiment(s) of the present disclosure.

FIG. 11 illustrates an example computing device, in accordance with embodiment(s) of the present disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

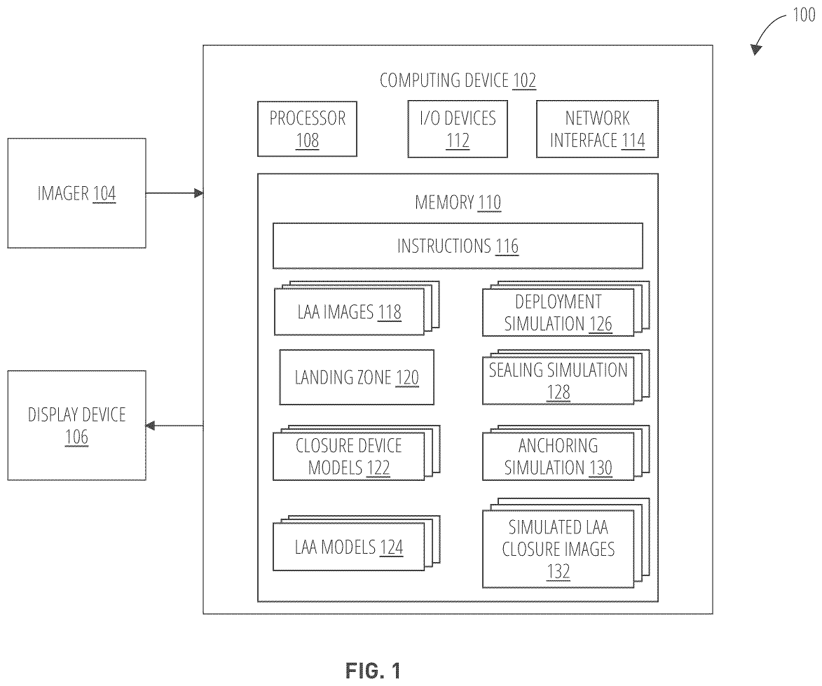

FIG. 1 illustrates a LAA closure planning system 100, in accordance with non-limiting example(s) of the present disclosure. In general, LAA closure planning system 100 is a system for planning an LAA closure using a closure device. For example, the Watchman FLX® by Boston Scientific Corporation® is an LAA closure device. Patients with non-valvular atrial fibrillation may be treated for an LAA closure, such as by implanting a closure device in the LAA to reduce the risk of thromboembolism from the LAA. The present disclosure can be used for pre-planning the procedure to close the LAA with such a device.

LAA closure planning system 100 includes a computing device 102. Optionally, LAA closure planning system 100 includes imager 104 and display device 106. In an example, computing device 102 can receive an image or a group of images representative of an anatomy of a patient's LAA. For example, computing device 102 can receive LAA images 118 from imager 104. In some embodiments, imager 104 can be a CT imager arranged to generate images comprising indication of (or representative of) the LAA of a patient. In part from the LAA images 118, computing device 102 can generate simulated LAA closure images 132, which comprise indications of simulated closure device deployments within the patient's LAA (e.g., the LAA represented in LAA images 118).

Although the disclosure uses CT imaging to describe illustrative embodiments, imager 104 can be any of a LAA imaging devices, such as, for example, a fluoroscopy imaging device, an ultrasound imaging device, a computed tomography (CT) imaging device, a magnetic resonance (MR) imaging device, a positron emission tomography (PET) imaging device, or a single-photon emission computed tomography (SPECT) imaging device.

Imager 104 can generate information elements, or data, including indications of LAA images 118. Computing device 102 is communicatively coupled to imager 104 and can receive the data including the indications of LAA images 118 from imager 104. In general, LAA images 118 can include indications of shape data and/or appearance data of the LAA. Shape data can include landmarks, surfaces, and boundaries of the three-dimensional surface of the LAA. With some examples, LAA images 118 can be constructed from two-dimensional (2D) or three-dimensional (3D) images of the LAA. In some embodiments, LAA images 118 can be a medical image stored in a medical imaging database and subjected to access controls for medical images. The term “image” is used herein for clarity of presentation and to imply that LAA images 118 represents the structure and anatomy of the LAA. However, it is to be appreciated that the term “image” is not to be limiting. That is, LAA images 118 may not be an image as conventionally used, or rather, an image viewable and interpretable by a human. For example, LAA images 118 can be a point cloud, a parametric model, or other morphological description of the anatomy of the LAA. Furthermore, LAA images 118 can be a single image, or a series of images.

In general, display device 106 can be a digital display arranged to receive rendered image data and display the data in a graphical user interface. Computing device 102 can be any of a variety of computing devices. In some embodiments, computing device 102 can be incorporated into and/or implemented by a console of display device 106. With some embodiments, computing device 102 can be a workstation or server communicatively coupled to imager 104 and/or display device 106. With still other embodiments, computing device 102 can be provided by a cloud based computing device, such as, by a computing as a service system accessibly over a network (e.g., the Internet, an intranet, a wide area network, or the like). Computing device 102 can include processor 108, memory 110, input and/or output (I/O) devices 112, and network interface 114.

The processor 108 may include circuitry or processor logic, such as, for example, any of a variety of commercial processors. In some examples, processor 108 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 108 may include graphics processing portions and may include dedicated memory, multiple-threaded processing and/or some other parallel processing capability. In some examples, the processor 108 may be an application specific integrated circuit (ASIC) or a field programmable integrated circuit (FPGA).

The memory 110 may include logic, a portion of which includes arrays of integrated circuits, forming non-volatile memory to persistently store data or a combination of non-volatile memory and volatile memory. It is to be appreciated, that the memory 110 may be based on any of a variety of technologies. In particular, the arrays of integrated circuits included in memory 120 may be arranged to form one or more types of memory, such as, for example, dynamic random access memory (DRAM), NAND memory, NOR memory, or the like.

I/O devices 112 can be any of a variety of devices to receive input and/or provide output. For example, I/O devices 112 can include, a keyboard, a mouse, a joystick, a foot pedal, a display (e.g., touch, non-touch, or the like) different from display device 106, a haptic feedback device, an LED, or the like.

Network interface 114 can include logic and/or features to support a communication interface. For example, network interface 114 may include one or more interfaces that operate according to various communication protocols or standards to communicate over direct or network communication links. Direct communications may occur via use of communication protocols or standards described in one or more industry standards (including progenies and variants). For example, network interface 114 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 114 can include logic and/or features to enable communication over a variety of wired or wireless network standards (e.g., 802.11 communication standards). For example, network interface 114 may be arranged to support wired communication protocols or standards, such as, Ethernet, or the like. As another example, network interface 114 may be arranged to support wireless communication protocols or standards, such as, for example, Wi-Fi, Bluetooth, ZigBee, LTE, 5G, or the like.

Memory 110 can include instructions 116, LAA images 118, landing zone 120, closure device models 122, LAA models 124, deployment simulation 126, sealing simulation 128, anchoring simulation 130, and simulated LAA closure images 132. During operation, processor 108 can execute instructions 116 to cause computing device 102 to receive LAA images 118 from imager 104 and landing zone 120 from a physician via input and/or output (I/O) devices 112. Processor 108 can further execute instructions 116 to identify a recommended size of closure device, simulate deployment, sealing, and anchoring of the recommended closure device. Further still, processor 108 can execute instructions 116 to generate simulated LAA closure images 132 and cause 132 to be displayed on display device 106.

In general, closure device models 122 and LAA models 124 can be finite elements and can be used with any of a variety of finite element methods (FEM), such as, for example, applied element method (AEM), augmented-FEM (A-FEM), generalized FEM, mixed FEM, variable polynomial, hpk-FEM, extended FEM (XFEM), scaled boundary FEM (SBFEM), smoothed FEM (SFEM), meshfree methods, discontinuous galerkin methods, stretched grid method, finite element limit analysis, loubignac iteration, crystal plasticity FEM (CPFEM), or virtual element method (VEM) to name a few.

With other examples, closure device models 122 and LAA models 124 can be machine learning models, and in particular models adapted for image recognition and processing, such as, for example, neural networks, convoluted neural networks, K nearest neighbor, decision trees, or support vector machines, to name a few.

In some examples, processor 108 can execute instructions 116 to solve the equations represented by closure device models 122 and LAA models 124 given the LAA images 118 and landing zone 120 (e.g., in the case that closure device models 122 and LAA models 124 are finite elements) to determine deployment simulation 126, sealing simulation 128 and anchoring simulation 130. In other examples, processor 108 can execute instructions 116 to generate an output from the closure device models 122 and LAA models 124 given LAA images 118 and landing zone 120 (e.g., in the case that closure device models 122 and LAA models 124 are machine learning models) to infer deployment simulation 126, sealing simulation 128, and anchoring simulation 130. With some examples, although not shown in this figure, one or more closure device models 122 and/or LAA models 124 can be provided for each “simulation” (e.g., deployment simulation 126, sealing simulation 128, or anchoring simulation 130.

Processor 108 can execute instructions 116 to generate (e.g., process, render, or the like) simulated LAA closure images 132 from deployment simulation 126, sealing simulation 128 and/or anchoring simulation 130.

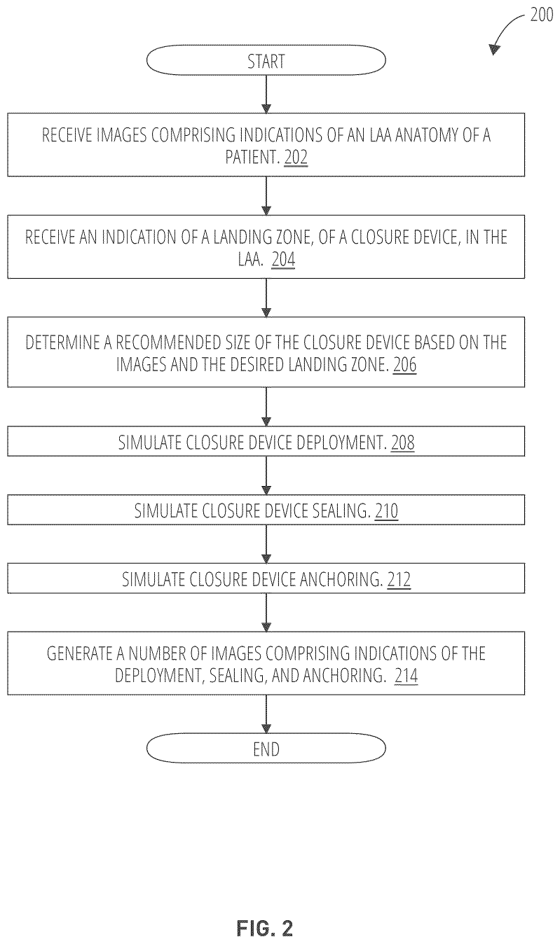

The above is described in greater detail below, such as, for example, in conjunction with 200 from FIG. 2. With some examples, LAA closure planning system 100 can be provided with just computing device 102. That is, LAA closure planning system 100 can include computing device 102 and a user of LAA closure planning system 100 can provide imager 104 and display device 106, which are compatible with computing device 102. In another example, LAA closure planning system 100 can include just instructions 116, closure device models 122, and LAA models 124, which can be executed by a comparable computing system (e.g., a cloud computing service, or the like) to generate simulated LAA closure images 132 as outlined herein.

It is noted that LAA closure planning system 100 includes custom components, which are specifically configured, programmed, and/or arranged to carry out the logic flows and methods detailed herein. For example, processor 108 can be preconfigured to execute a FEM using finite elements such as, for example, closure device models 122 and LAA models 124. As another example, processor 108 could be preconfigured to infer output from a machine learning model (e.g., closure device models 122 and LAA models 124).

Further, closure device models 122 and LAA models 124 are preprogrammed specific to the detailed logic flows, methods, and techniques discussed herein and are not conventional or general purpose computing components.

FIG. 2 illustrates logic flow 200, according to some embodiments of the present disclosure. Logic flow 200 can be implemented to generate pre-procedure planning images for an LAA closure procedure based on pre-procedure imaging. For example, logic flow 200 can be provided to generate simulated LAA closure images 132 given LAA images 118. Although logic flow 200 is described with reference to LAA closure planning system 100 and FIG. 1, examples are not limited by this and logic flow 200 could be implemented by a system having components that depicted in FIG. 1.

Logic flow 200 can being at block 202. At block 202 “receive images comprising indications of an LAA anatomy of a patient” images comprising indications of an LAA anatomy of a patient are received. For example, LAA images 118 can be received by computing device 102 from imager 104. Processor 108 can execute instructions 116 to receive LAA images 118 from imager 104.

Continuing to block 204 “receive an indication of a landing zone, of a closure device, in the LAA” a landing site within the patient LAA is received. For example, landing zone 120 can be received by computing device 102 (e.g., from a physician via input and/or output (I/O) devices 112). As a specific example, processor 108 can execute instructions 116 to cause LAA images 118 to be displayed on display device 106 and landing zone 120 can be received from a physician, such as, for example, via a touch screen gesture, via a mouse input, or the like.

Continuing to block 206 “determine a recommended size of the closure device based on the images and the desired landing zone” a recommended size of the closure device is determined based on the images and the landing zone. With some examples, several closure devices can be available and represented by closure device models 122, such as, for example, closure device having a size between 20 and 40 millimeters (mm). With some examples, processor 108 can execute instructions 116 to identify a size based on the ostium diameter of the LAA represented in LAA images 118 at the landing zone 120. In some embodiments, at block 206, processor 108 can execute instructions 116 to determine a recommended size or an initial starting size for simulation, where the final recommended size may be based on the simulation as described herein (e.g., at block 212 and block 214, or the like).

In some examples, the recommended closure device is identified based on determining the maximum LAA diameter at the landing zones and selecting a closure device that has a diameter like the maximum LAA diameter at the landing zone when compressed 10 to 30 percent.

Continuing to block 208 “simulate closure device deployment” the deployment of the determined closure device size (and optionally one or more other sizes) in the LAA of the patient is simulated. For example, deployment simulation 126 is generated based on LAA images 118, landing zone 120, closure device models 122, and LAA models 124. More particularly, processor 108 can execute instructions 116 to generate deployment simulation 126 from LAA images 118, landing zone 120, closure device models 122, and/or LAA models 124. As a specific example, processor 108 can execute instructions 116 to solve equations represented by finite elements of closure device models 122 and LAA models 124 to simulate how the determined size of the closure device responds (e.g., expands and deploys, or the like) to the anatomy of the LAA represented in the LAA images 118 given the landing zone 120. Further, with some embodiments, processor 108 can execute instructions 116 to solve equations represented by finite elements of closure device models 122 and LAA models 124 to simulate how alternative sizes (e.g., a larger and a smaller closure device size, all closure device sizes, or the like) of the closure device respond (e.g., expands and deploys, or the like) to the anatomy of the LAA represented in the LAA images 118 given the landing zone 120. With some embodiments, processor 108 can execute instructions 116 to generate a surface mesh (e.g., a stereolithography (STL) mesh, or the like) of the LAA from the LAA images 118, which mesh is then converted into an FEA mesh.

Continuing to block 210 “simulate closure device sealing” the sealing of the determined closure device size in the LAA of the patient is simulated. For example, sealing simulation 128 is generated based on LAA images 118, landing zone 120, closure device models 122, LAA models 124, and deployment simulation 126. More particularly, processor 108 can execute instructions 116 to generate sealing simulation 128 from LAA images 118, landing zone 120, closure device models 122, LAA models 124, and/or deployment simulation 126. As a specific example, processor 108 can execute instructions 116 to solve equations represented by finite elements of closure device models 122 and LAA models 124 to simulate how the expanded and deployed closure device (e.g., having the determined size and optionally alternative sizes) seals against (or forms a seal with) the anatomy of the LAA represented in the LAA images 118 given the landing zone 120 and deployment simulation 126.

Continuing to block 212 “simulate closure device anchoring” the anchoring of the determined closure device size in the LAA of the patient is simulated. For example, anchoring simulation 130 is generated based on LAA images 118, landing zone 120, closure device models 122, LAA models 124, and deployment simulation 126. More particularly, processor 108 can execute instructions 116 to generate anchoring simulation 130 from LAA images 118, landing zone 120, closure device models 122, LAA models 124, and/or deployment simulation 126. As a specific example, processor 108 can execute instructions 116 to solve equations represented by finite elements of closure device models 122 and LAA models 124 to simulate how the expanded and deployed closure device (e.g., having the determined size and optionally alternative sizes) anchors in the anatomy of the LAA represented in the LAA images 118 given the landing zone 120 and deployment simulation 126.

Continuing to block 214 “generate a number of images comprising indications of the deployment, sealing, and anchoring” images comprising indications of the deployment, sealing, and anchoring are generated. For example, simulated LAA closure images 132 can be generated from LAA images 118, landing zone 120, deployment simulation 126, sealing simulation 128, and/or anchoring simulation 130. Processor 108 can execute instructions 116 to generate simulated LAA closure images 132 from LAA images 118, landing zone 120, deployment simulation 126, sealing simulation 128, and/or anchoring simulation 130. With some examples, processor 108 executes instructions 116 to generate a 132 based on overlaying the LAA represented in LAA images 118 with the simulated deployed closure device, the simulated sealing, and/or the simulated anchoring. As noted above, with some embodiments, simulations for multiple (e.g., the recommended closure device size and alternative closure device sizes) are simulated. As such, simulated LAA closure images 132 can include multiple images representative of the various simulations for the various closure device sizes.

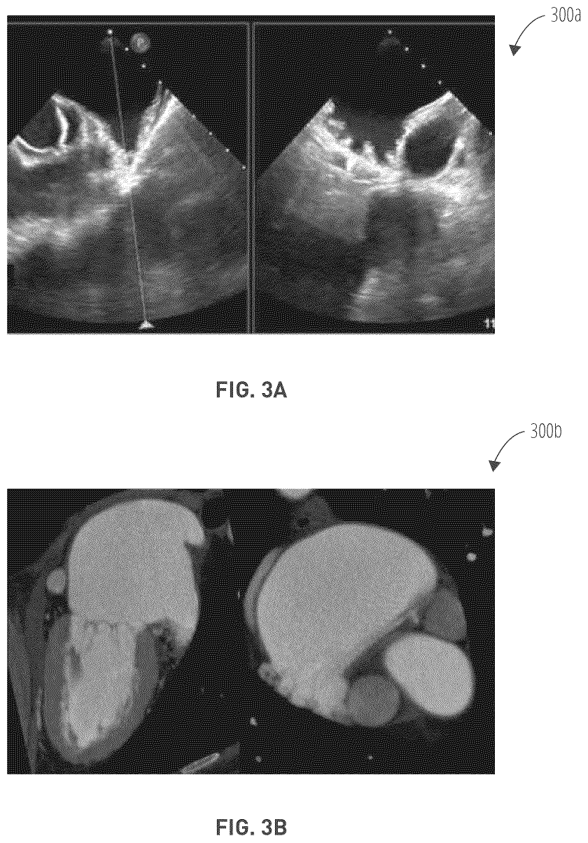

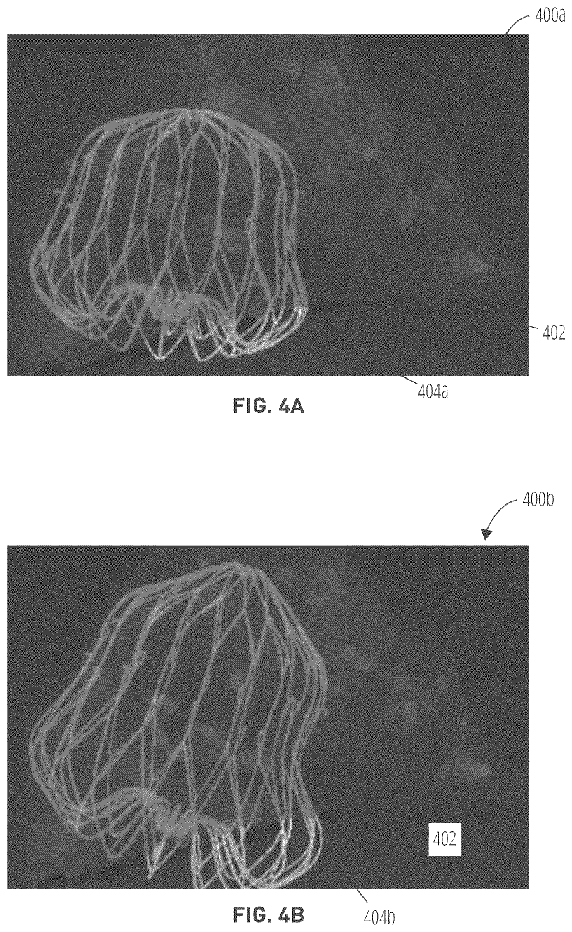

FIG. 3A and FIG. 3B illustrate LAA image 300a and LAA image 300b, respectively. LAA image 300a and LAA image 300b are representative images of an LAA, which can be like LAA images 118 described above. For example, LAA image 300a and LAA image 300b are CT images of an LAA of a patient, which can be received by computing device 102 from imager 104 as outlined above. As noted, with some embodiments, LAA images 118 can be a series of images of an LAA of a patient (e.g., multiple views, angles, or the like). An anatomy of the LAA and the model from LAA models 124 can be determined based on the multiple LAA images 118. For example, points within the images that are representative of specific LAA anatomy can be identified and a models from LAA models 124 having similar (e.g., the same, within a threshold distance, or the like) points can be selected for use.

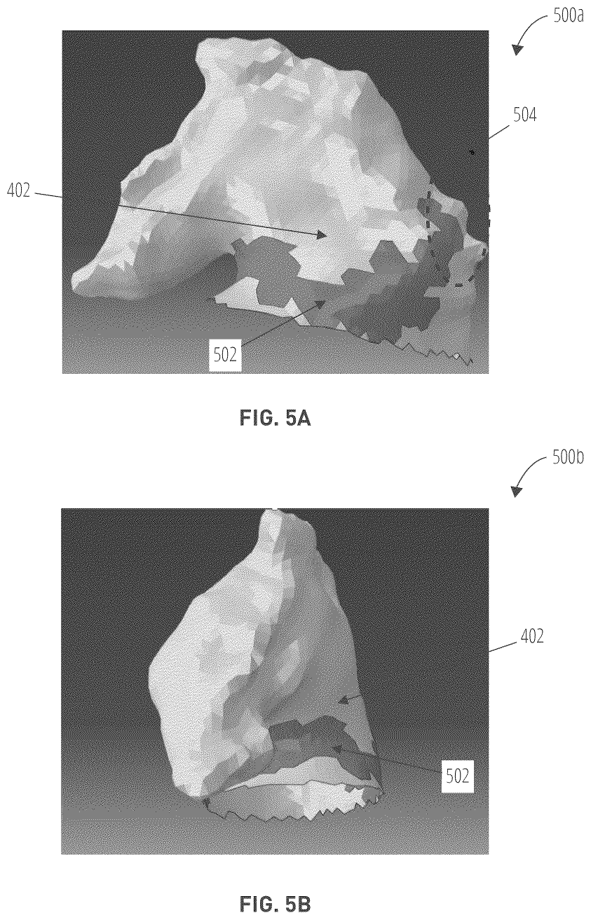

FIG. 4A and FIG. 4B illustrate simulated deployment image 400a and simulated deployment image 400b, respectively. Simulated deployment image 400a and simulated deployment image 400b are representative of images of a deployment of a closure device in an LAA, which can be like ones of simulated LAA closure images 132 described above. For example, simulated deployment image 400a comprises LAA 402, which is a graphical representation of the patients LAA (e.g., based on LAA images 118) as well as a graphical representation of the deployed closure device 404a of the recommended size. Similarly, simulated deployment image 400a comprises LAA 402 as well as a graphical representation of the deployed closure device 404b of an alternative size.

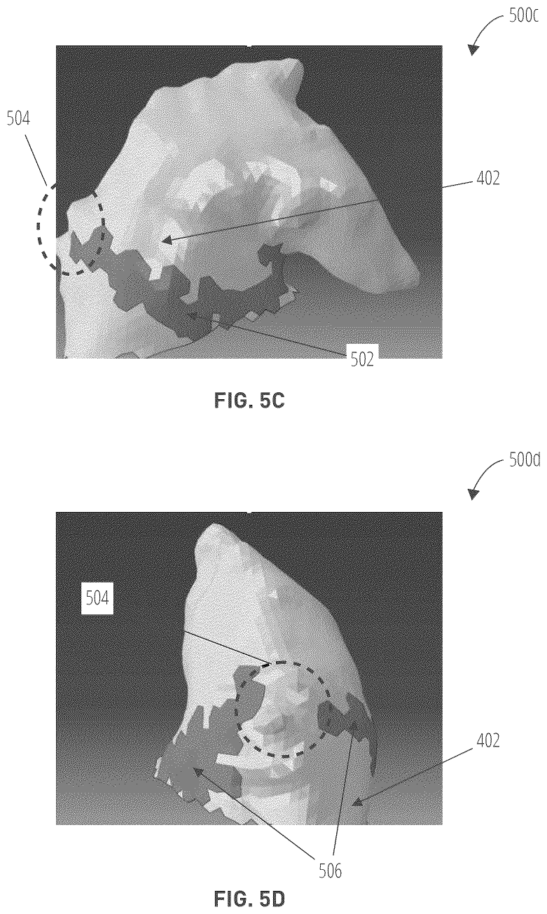

FIG. 5A, FIG. 5B, FIG. 5C, and FIG. 5D illustrate simulated sealing image 500a, simulated sealing image 500b, simulated sealing image 500c, and simulated sealing image 500d, respectively. Simulated sealing images 500a, 500b, 500c, and 500d are representative of a sealing, or contact, of the closure device against the LAA anatomy. For example, simulated sealing images 500a, 500b, 500c, and 500d comprise LAA 402 as well as a graphical representations of the sealing, or contact, of the closure device (of the recommended size, or an alternative size) against the perimeter of the LAA. With some examples, this can be shown by several views (e.g., 4 in this case). These images comprise contact surface 502, which is indicative of (e.g., a representation of) sealing of the closure device against the LAA as well as leak path 504, which is indicative of (e.g., representative of) a gap in the contact of the closure device and the LAA 402.

With some examples, simulated sealing images 500a, 500b, 500c, and 500d can be provided as part of simulated LAA closure images 132. In other examples, simulated sealing images 500a, 500b, 500c, and 500d can be represented in a 3D model, which can be manipulated (e.g., rotated, or the like) to show all angles of the LAA 402 including the contact surface 502 and any leak paths 504. As another example, simulated LAA closure images 132 can comprise a video comprising frames of images like simulated sealing images 500a, 500b, 500c, and 500d to provide a graphical representation of the perimeter of the LAA 402 showing contact surface 502 and leak paths 504.

In some examples, simulated sealing images can be generated based on a two-dimensional (2D) cross-section map at the desired landing zone with the deployed closure device. In such a 2D cross-section map, the contours of the LAA anatomy and the closure device can be identified or indicated while any “gaps” between the LAA anatomy and the closure device can be highlighted (e.g., indicated with color, a graphic, or the like).

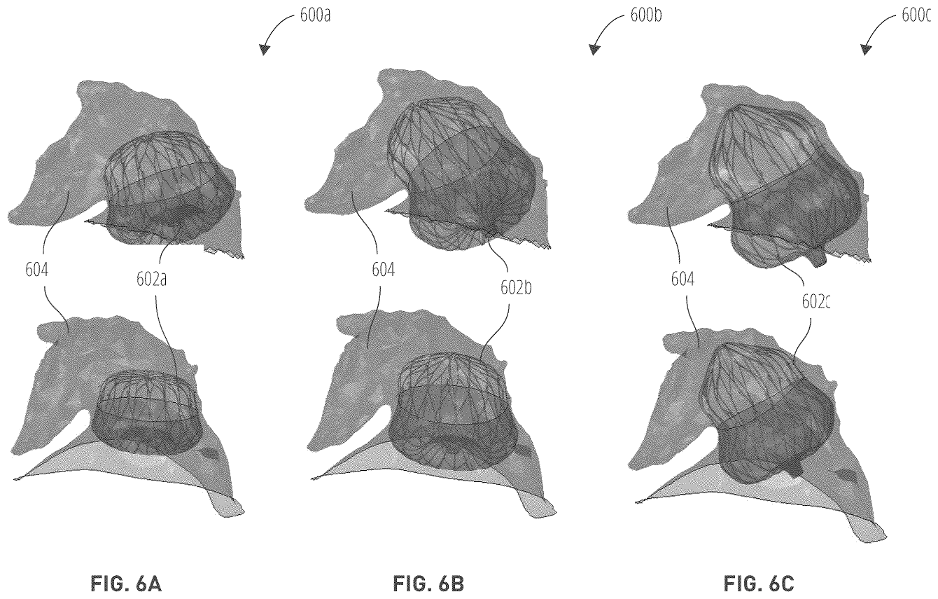

FIG. 6A illustrates simulated anchor engagement image 600. Simulated anchor engagement image 600 comprises an indication of the deployed closure device of the recommended size (or an alternative size). It is to be appreciated that LAA closure devices often include anchors to engage tissue of the LAA that “hold” the closure device in place until LAA tissue grows into and around the closure device. For example, simulated anchor engagement image 600 shows deployed closure device 404a having anchors 602. Further, simulated anchor engagement image 600 comprises a graphical representation of (or indication of) the engagement of the tip of each anchor 602. For example, simulated anchor engagement image 600 comprises a color map showing the penetration distance of each anchor 602 into tissue of the LAA 402. With other examples, simulated anchor engagement image 600 can comprise a color map showing the contact force of each anchor 602 against the tissue of the LAA 402.

FIG. 6B illustrates exploded view 604 from simulated anchor engagement image 600 of FIG. 6A in larger detail. As can be more clearly seen from this figure, anchor engagements 606 for each anchor 602 of deployed closure device 404a are shown as a color map (or heat map) indicative of (or representative of) the engagement (e.g., penetration depths, contact force, or the like) of the anchors 602 with tissue of the LAA 402.

With some examples, simulated sealing images 500a, 500b, 500c, and 500d can be provided as part of simulated LAA closure images 132. In other examples, simulated sealing images 500a, 500b, 500c, and 500d can be represented in a 3D model, which can be manipulated (e.g., rotated, or the like) to show all angles of the LAA 402 including the contact surface 502 and any leak paths 504. As another example, simulated LAA closure images 132 can comprise a video comprising frames of images like simulated sealing images 500a, 500b, 500c, and 500d to provide a graphical representation of the perimeter of the LAA 402 showing contact surface 502 and leak paths 504.

In some embodiments, processor 108 can execute instructions 116 to determine an optimal size for the LAA closure device based on several simulated sealing images. FIG. 6A, FIG. 6B, and FIG. 6C, illustrate pairs of sealing images 600a, 600b, and 600c, respectively. These pairs of sealing images depict simulated sealing of LAA closure devices 602a, 602b, and 602c against a LAA anatomy 604. From these pairs of images or from the FAE simulation and image data, processor 108 can execute instructions 116 to determine a path loop (e.g., around the LAA closure devices 602a, etc.) with the minimum gap area. This minimum gap area and/or minimum gap path can be reported to the user (e.g., via a display device 106, or the like). With some embodiments, processor 108 can execute instructions 116 to determine the minimum gap area based on a hydraulic diameter or flow resistance using the simulated sealing.

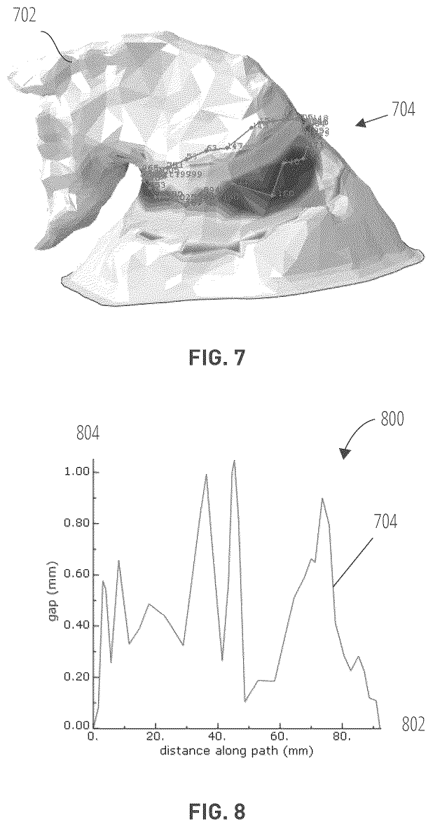

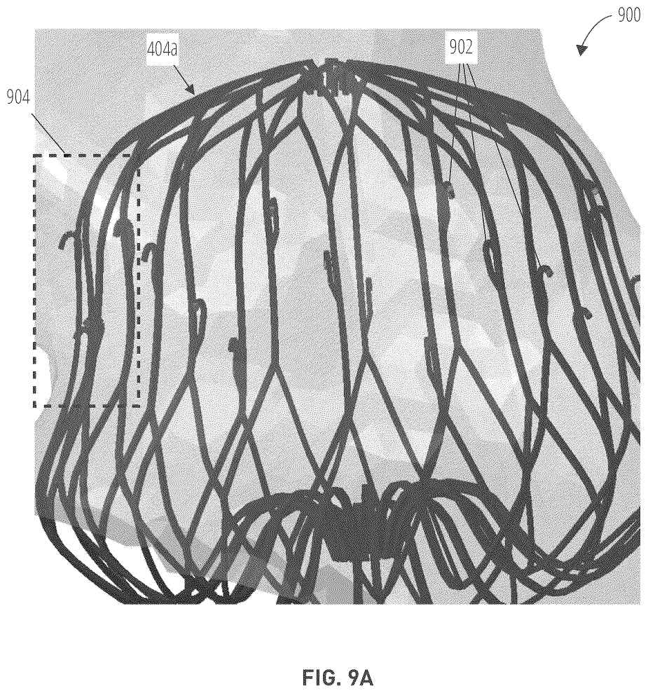

For example, FIG. 7 illustrates a LAA anatomy 702 and a leak path 704 based on a simulated deployed closure device (not shown). FIG. 8 illustrates a graph 800 showing the gap of leak path 704 plotted on the 804 against the distance along leak path 704 on the x axis 802. With some examples, the total leak path 704 gap can be integrated (e.g., area under graph 800, or the like) and the closure device with the smallest total leak path gap can be identified as the optimal or recommended closure device size. In some embodiments, the integrated leak path gap can be normalized for the closure device size (e.g., 35 mm, 37 mm, 40 mm, or the like).

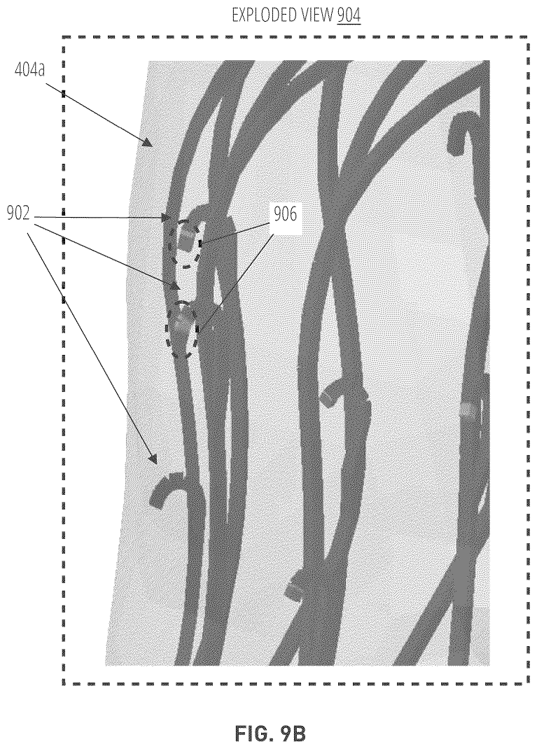

FIG. 9A illustrates simulated anchor engagement image 900. Simulated anchor engagement image 900 comprises an indication of the deployed closure device of the recommended size (or an alternative size). It is to be appreciated that LAA closure devices often include anchors to engage tissue of the LAA that “hold” the closure device in place until LAA tissue grows into and around the closure device. For example, simulated anchor engagement image 900 shows deployed closure device 404a having anchors 902. Further, simulated anchor engagement image 900 comprises a graphical representation of (or indication of) the engagement of the tip of each anchor 902. For example, simulated anchor engagement image 900 comprises a color map showing the penetration distance of each anchor 902 into tissue of the LAA 402. With other examples, simulated anchor engagement image 900 can comprise a color map showing the contact force of each anchor 902 against the tissue of the LAA 402.

FIG. 9B illustrates exploded view 904 from simulated anchor engagement image 900 of FIG. 9A in larger detail. As can be more clearly seen from this figure, anchor engagements 906 for each anchor 902 of deployed closure device 404a are shown as a color map (or heat map) indicative of (or representative of) the engagement (e.g., penetration depths, contact force, or the like) of the anchors 902 with tissue of the LAA 402. In some embodiments, the penetration depth can be identified and reported (e.g., to flag areas of potential preformation, or the like). In another example, the number of anchors engaged (e.g., greater than a threshold level, or the like) can be identified and reported (e.g., to indicate the potential stability of the deployment, or the like).

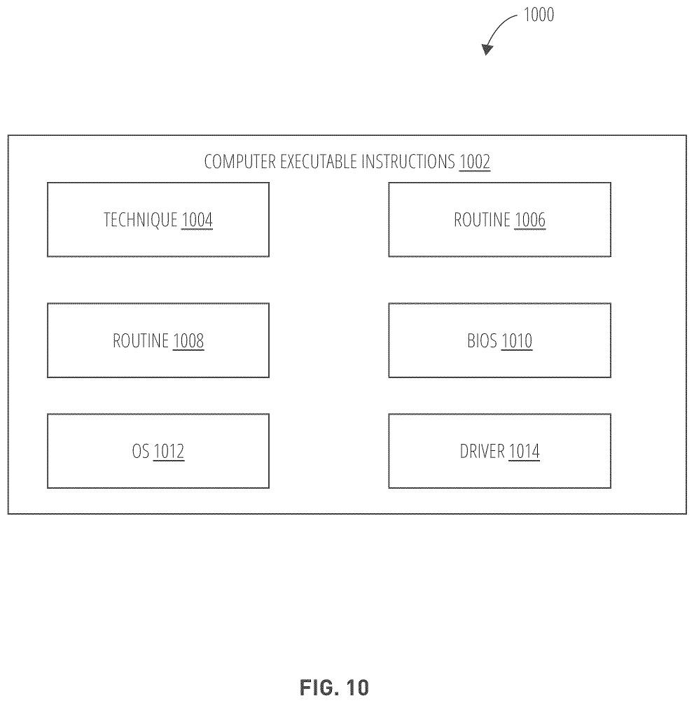

FIG. 10 illustrates computer-readable storage medium 1000. Computer-readable storage medium 1000 may comprise any non-transitory computer-readable storage medium or machine-readable storage medium, such as an optical, magnetic or semiconductor storage medium. In various embodiments, computer-readable storage medium 1000 may comprise an article of manufacture. In some embodiments, 700 may store computer executable instructions 1002 with which circuitry (e.g., processor 108, or the like) can execute. For example, computer executable instructions 1002 can include instructions to implement operations described with respect to logic flow 200, instructions 116, closure device models 122, and/or LAA models 124. Examples of computer-readable storage medium 1000 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1002 may include any suitable type of code, such as source code, compiled code, interpreted code, executable code, static code, dynamic code, object-oriented code, visual code, and the like.

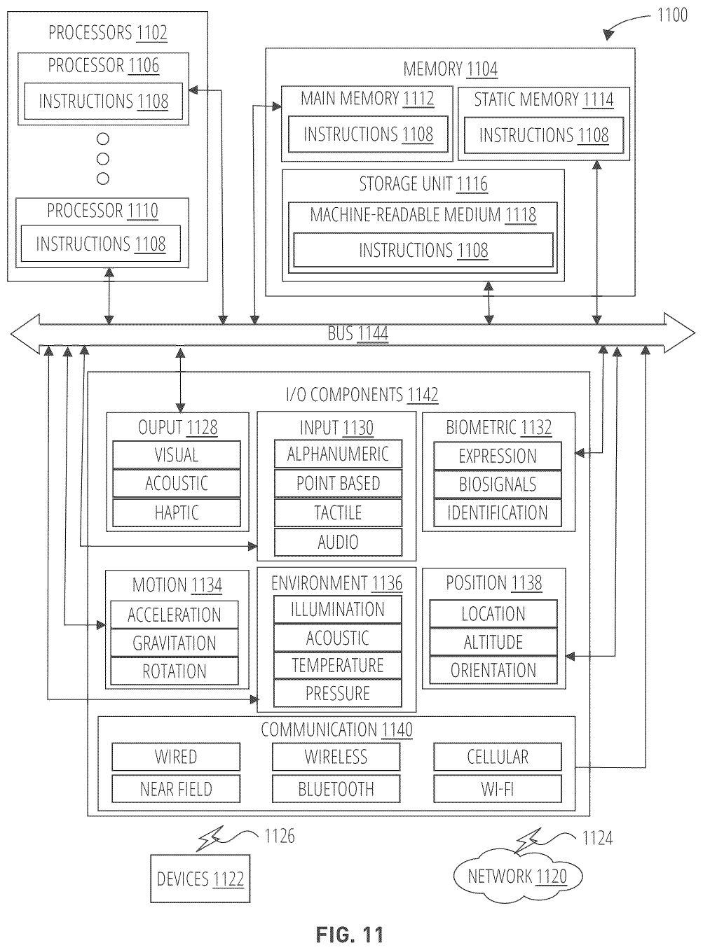

FIG. 11 illustrates a diagrammatic representation of a machine 1100 in the form of a computer system within which a set of instructions may be executed for causing the machine to perform any one or more of the methodologies discussed herein. More specifically, FIG. 11 shows a diagrammatic representation of the machine 1100 in the example form of a computer system, within which instructions 1108 (e.g., software, a program, an application, an applet, an app, or other executable code) for causing the machine 1100 to perform any one or more of the methodologies discussed herein may be executed. For example, the instructions 1108 may cause the machine 1100 to execute logic flow 200 of FIG. 2, or the like. More generally, the instructions 1108 may cause the machine 1100 to provide pre-procedure planning for an LAA closure procedure, such as, for example receiving LAA images 118 and generating deployment simulation 126, sealing simulation 128, anchoring simulation 130 and/or simulated LAA closure images 132 as outlined above.

The instructions 1108 transform the general, non-programmed machine 1100 into a particular and specifically programmed machine 1100 configured to carry out the described and illustrated functions in a specific manner. In alternative embodiments, the machine 1100 operates as a standalone device or may be coupled (e.g., networked) to other machines. In a networked deployment, the machine 1100 may operate in the capacity of a server machine or a client machine in a server-client network environment, or as a peer machine in a peer-to-peer (or distributed) network environment. The machine 1100 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1108, sequentially or otherwise, that specify actions to be taken by the machine 1100. Further, while only a single machine 1100 is illustrated, the term “machine” shall also be taken to include a collection of machines 200 that individually or jointly execute the instructions 1108 to perform any one or more of the methodologies discussed herein.

The machine 1100 may include processors 1102, memory 1104, and I/O components 1142, which may be configured to communicate with each other such as via a bus 1144. In an example embodiment, the processors 1102 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1106 and a processor 1110 that may execute the instructions 1108. The term “processor” is intended to include multi-core processors that may comprise two or more independent processors (sometimes referred to as “cores”) that may execute instructions contemporaneously. Although FIG. 11 shows multiple processors 1102, the machine 1100 may include a single processor with a single core, a single processor with multiple cores (e.g., a multi-core processor), multiple processors with a single core, multiple processors with multiples cores, or any combination thereof.

The memory 1104 may include a main memory 1112, a static memory 1114, and a storage unit 1116, both accessible to the processors 1102 such as via the bus 1144. The main memory 1104, the static memory 1114, and storage unit 1116 store the instructions 1108 embodying any one or more of the methodologies or functions described herein. The instructions 1108 may also reside, completely or partially, within the main memory 1112, within the static memory 1114, within machine-readable medium 1118 within the storage unit 1116, within at least one of the processors 1102 (e.g., within the processor's cache memory), or any suitable combination thereof, during execution thereof by the machine 1100.

The I/O components 1142 may include a wide variety of components to receive input, provide output, produce output, transmit information, exchange information, capture measurements, and so on. The specific I/O components 1142 that are included in a particular machine will depend on the type of machine. For example, portable machines such as mobile phones will likely include a touch input device or other such input mechanisms, while a headless server machine will likely not include such a touch input device. It will be appreciated that the I/O components 1142 may include many other components that are not shown in FIG. 11. The I/O components 1142 are grouped according to functionality merely for simplifying the following discussion and the grouping is in no way limiting. In various example embodiments, the I/O components 1142 may include output components 1128 and input components 1130. The output components 1128 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1130 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1142 may include biometric components 1132, motion components 1134, environmental components 1136, or position components 1138, among a wide array of other components. For example, the biometric components 1132 may include components to detect expressions (e.g., hand expressions, facial expressions, vocal expressions, body gestures, or eye tracking), measure bio-signals (e.g., blood pressure, heart rate, body temperature, perspiration, or brain waves), identify a person (e.g., voice identification, retinal identification, facial identification, fingerprint identification, or electroencephalogram-based identification), and the like. The motion components 1134 may include acceleration sensor components (e.g., accelerometer), gravitation sensor components, rotation sensor components (e.g., gyroscope), and so forth. The environmental components 1136 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1138 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1142 may include communication components 1140 operable to couple the machine 1100 to a network 1120 or devices 1122 via a coupling 1124 and a coupling 1126, respectively. For example, the communication components 1140 may include a network interface component or another suitable device to interface with the network 1120. In further examples, the communication components 1140 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1122 may be another machine or any of a wide variety of peripheral devices (e.g., a peripheral device coupled via a USB).

Moreover, the communication components 1140 may detect identifiers or include components operable to detect identifiers. For example, the communication components 1140 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1140, such as location via Internet Protocol (IP) geolocation, location via Wi-Fi® signal triangulation, location via detecting an NFC beacon signal that may indicate a particular location, and so forth.

The various memories (i.e., memory 1104, main memory 1112, static memory 1114, and/or memory of the processors 1102) and/or storage unit 1116 may store one or more sets of instructions and data structures (e.g., software) embodying or utilized by any one or more of the methodologies or functions described herein. These instructions (e.g., the instructions 1108), when executed by processors 1102, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1120 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1120 or a portion of the network 1120 may include a wireless or cellular network, and the coupling 1124 may be a Code Division Multiple Access (CDMA) connection, a Global System for Mobile communications (GSM) connection, or another type of cellular or wireless coupling. In this example, the coupling 1124 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1108 may be transmitted or received over the network 1120 using a transmission medium via a network interface device (e.g., a network interface component included in the communication components 1140) and utilizing any one of several well-known transfer protocols (e.g., hypertext transfer protocol (HTTP)). Similarly, the instructions 1108 may be transmitted or received using a transmission medium via the coupling 1126 (e.g., a peer-to-peer coupling) to the devices 1122. The terms “transmission medium” and “signal medium” mean the same thing and may be used interchangeably in this disclosure. The terms “transmission medium” and “signal medium” shall be taken to include any intangible medium that can store, encoding, or carrying the instructions 1108 for execution by the machine 1100, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A method of pre-planning a left atrial appendage (LAA) closure, comprising:
receiving, at a computing device, images comprising indication of an LAA of a patient;
receiving, at the computing device, an indication of a landing zone of a closure device;
simulating, by the computing device, a deployment of the closure device in the LAA of the patient;
simulating, by the computing device, a sealing of the deployed closure device against the LAA of the patient;
simulating, by the computing device, an anchoring of the closure device in the LAA of the patient; and
generating, by the computing device, a graphical representation of the sealing and anchoring of the closure device in the LAA of the patient.

2. The method of claim 1, comprising generating a graphical representation of the deployed closure device in the LAA of the patient.

3. The method of claim 2, generating a graphical representation of the deployed closure device in the LAA of the patient comprising overlaying the simulated deployed closure device with a graphical representation of the LAA of the patient.

4. The method of claim 1, comprising determining a recommended size of the closure device based on the received images.

5. The method of claim 4, comprising:
identifying a diameter of the ostium of the LAA of the patient based on the received images;
selecting from a set of closure device sizes a closure device size within a threshold expanded diameter to the diameter of the ostium; and
identifying the selected closure device size as the recommended closure device.

6. The method of claim 4, comprising simulating the deployment of the closure device in the LAA of the patient for a closure device having the recommended size.

7. The method of claim 6, comprising simulating a deployment of the closure device in the LAA of the patient for a closure device having a size different from the recommended size.

8. The method of claim 7, comprising generating a graphical representation of the deployed closure device in the LAA of the patient comprising overlaying the simulated deployed closure device having the recommended size with the graphical representation of the LAA of the patient and overlaying the simulated deployed closure device having a different size with the graphical representation of the LAA of the patient.

9. The method of claim 1, generating the graphical representation of the sealing of the deployed closure device in the LAA of the patient comprising generating a plurality of images comprising an indication of the LAA of the patient and an indication of a contact surface, the contact surface representative of a contact between the deployed closure device and the LAA of the patient.

10. The method of claim 9, the plurality of images comprising the indication of the LAA of the patient, the indication of the contact surface and an indication of a leak path.

11. The method of claim 10, comprising generating a rotatable model from the plurality of images.

12. The method of claim 1, generating the graphical representation of the anchoring of the closure device in the LAA of the patient comprising generating an image of the simulated deployed closure device comprising indications of anchors of the closure device and a representation of an engagement of the anchors with tissue of the LAA of the patient.

13. The method of claim 12, wherein the representation of the engagement of the anchors comprises a color map.

14. A computing system, comprising:
a processor; and
memory comprising instructions, which when executed by the processor cause the computing system to:
receive images comprising indication of an LAA of a patient;
receive an indication of a landing zone of a closure device;
simulate a deployment of the closure device in the LAA of the patient;
simulate a sealing of the deployed closure device against the LAA of the patient;
simulate an anchoring of the closure device in the LAA of the patient; and
generate a graphical representation of the sealing and anchoring of the closure device in the LAA of the patient.

15. The computing system of claim 14, the instruction, when executed by the processor, cause the computing system to generate a graphical representation of the deployed closure device in the LAA of the patient.

16. The computing system of claim 15, the instruction, when executed by the processor, cause the computing system to generate a graphical representation of the deployed closure device in the LAA of the patient comprising overlaying the simulated deployed closure device with a graphical representation of the LAA of the patient.

17. The computing system of claim 14, the instruction, when executed by the processor, cause the computing system to determine a recommended size of the closure device based on the received images.

18. A computer readable storage medium comprising instructions, which when executed by a processor of a computing device cause the processor to:
receive images comprising indication of an LAA of a patient;
receive an indication of a landing zone of a closure device;
identifying a diameter of the ostium of the LAA of the patient based on the received images;
selecting from a set of closure device sizes a closure device size within a threshold expanded diameter to the diameter of the ostium;
identifying the selected closure device size as the closure device;
simulate a deployment of the closure device in the LAA of the patient;
simulate a sealing of the deployed closure device against the LAA of the patient;
simulate an anchoring of the closure device in the LAA of the patient; and
generate a graphical representation of the sealing and anchoring of the closure device in the LAA of the patient.

19. The computer readable storage medium of claim 18, the instruction, when executed by the processor, cause the computing system to simulate the deployment of the closure device in the LAA of the patient for a closure device having the recommended size.

20. The computer readable storage medium of claim 19, the instruction, when executed by the processor, cause the computing system to simulate a deployment of the closure device in the LAA of the patient for a closure device having a size different from the recommended size.
